(19) United States
(12) Patent Application Publication
(10) Pub. No.: US 2022/0233815 A1
(43) Pub. Date: Jul. 28, 2022

(54) CATHETER CONFIGURED TO MEASURE A FORCE ACTING ON THE CATHETER

(71) Applicant: VascoMed GmbH, Binzen (DE)

(72) Inventor: Robert BISCHOFF, Schliengen (DE)

(73) Assignee: VascoMed GmbH, Binzen (DE)

(21) Appl. No.: 17/615,712

(22) PCT Filed: Jun. 3, 2020
(86) PCT No.: PCT/EP2020/065321

§ 371 Date: Dec. 1, 2021

(30) Foreign Application Priority Data
Jun. 6, 2019 (EP) 19178634.2

(51) Int. Cl. A61M 25/00; A61M 25/09; A61B 18/14

(52) U.S. Cl. CPC A61M 25/0071; A61M 25/09; A61M 25/0053; A61B 2018/00077; A61M 2025/0002; A61M 2025/09183; A61M 2205/3306; A61B 18/14

(57) ABSTRACT

The disclosure relates to a catheter, comprising: an elongated shaft body extending along a longitudinal axis and having a distal end portion connected to a catheter tip at a distal end of the catheter, wherein the shaft body comprises a first lumen extending along the longitudinal axis, and an optical fiber for measuring a force, wherein the optical fiber extends in the first lumen and comprises at least a first Bragg grating arranged in in the distal end portion of the shaft body. The distal end portion of the shaft body encloses at least a first stiffening element, wherein the first stiffening element extends along the longitudinal axis for stiffening the distal end portion of the shaft body.

### CROSS-REFERENCE TO RELATED APPLICATIONS

This application is the United States national phase under 35 U.S.C. § 371 of PCT International Patent Application No. PCT/EP2020/065321, filed on Jun. 3, 2020, which claims the benefit of European Patent Application No. 19178634.2, filed on Jun. 6, 2019, the disclosures of which are hereby incorporated by reference herein in their entireties.

### TECHNICAL FIELD

The present disclosure relates to a catheter.

Such a catheter may comprise electrodes arranged on a distal end portion of a shaft body of the catheter in order to apply energy to tissue of a patient, particularly for ablation of patient tissue.

Such catheters can be configured to measure a force acting on the catheter tip by using an elongated optical fiber comprising at least one fiber Bragg grating formed in a portion of the optical fiber.

### BACKGROUND

Particularly, WIPO Publication No. WO 2016/149819 A1 describes a catheter comprising strain sensors that are connected to overlapping tubular members.

However, tubular members may bear the risk of rendering the distal end portion of the shaft of the catheter too stiff for flexible guidance of the tip of the catheter in case of certain applications, particularly in case of difficult to reach heart areas of a patient.

Therefore, one problem to be solved by the present invention is to provide a catheter capable of measuring a force acting on the catheter while achieving flexible guiding of the catheter tip at the same time.

At least this problem is solved by a catheter having the features of claim 1. Further embodiments are stated in the dependent and other claims and are described below.

The present disclosure is directed toward overcoming one or more of the above-mentioned problems, though not necessarily limited to embodiments that do.

### SUMMARY

A catheter is disclosed, comprising:
an elongated shaft body extending along a longitudinal axis and having a distal end portion connected to a catheter tip at a distal end of the catheter, wherein the shaft body comprises a first lumen extending along the longitudinal axis, and
an optical fiber for measuring a force wherein the optical fiber extends in the first lumen and comprises at least a first Bragg grating, wherein particularly the first lumen extends along the longitudinal axis.

The distal end portion of the shaft body encloses at least a first stiffening element, wherein the first stiffening element extends along the longitudinal axis for stiffening the distal end portion of the shaft body. In contrast to the prior art, the catheter does not comprise (or is free of) a metallic tubular force transducer arranged in the distal end portion of the shaft body.

The first stiffening element may be in the form of an elongated wire strand, an elongated wire braid, an elongated tubing (e.g. comprising a plastic material or be made of a plastic material) or a flat spring.

The present invention has the advantage that a rigid (metallic) force transducer can be completely replaced by components such as e.g. tubes, wire strands or braids, or lumens positioned one above the other within a flexible are of the shaft body. The position of these components in relation to one another allow an efficient force measurement using the optical fiber. At the same time, the design of the catheter is simplified. Furthermore, the entire distal end portion of the shaft body can be reversibly deformed, so that the catheter can be easily guided through standard locks.

In the framework of the present disclosure, the distal end of the catheter is formed by the catheter tip, wherein the catheter is inserted with the catheter tip ahead. At the proximal end, the catheter may comprise a handle for manually holding and operating the catheter.

Particularly, the Bragg grating used for force measurement is an optical interference filter inscribed in the optical fiber. Particularly, wavelengths of light coupled into the optical fiber that are within a filter bandwidth of the Bragg grating around the Bragg wavelength of the Bragg grating are reflected. The reflected wavelength shifts with the relative strain of the optical fiber at the location of the fiber Bragg grating. This allows measuring strain (or a force) acting on the optical fiber by measuring and analyzing the reflected wavelength shifts.

According to an embodiment, the distal end portion of the shaft body encloses a second stiffening element, wherein the second stiffening element extends along the longitudinal axis for stiffening the distal end portion of the shaft body. The second stiffening element may be in the form of an elongated wire strand, an elongated wire braid, an elongated tubing (e.g. comprising a plastic material or be made of a plastic material) or a flat spring.

Particularly, in an embodiment, the first and the second stiffening elements merely extend in the distal end portion of the shaft body. Particularly, the stiffening elements may be inserted in receptacles of the distal end portion shaft body via openings provided in the shaft body.

Furthermore, according to a preferred embodiment, the optical fiber comprises a second Bragg grating and a third Bragg grating for measuring said force acting on the catheter tip. This allows one to sense all force components in three dimensions. Particularly, the Bragg gratings are all located one after the other in the region of the distal end portion of the shaft body and are particularly spaced apart from one another.

Particularly, according to an embodiment, each Bragg grating comprises different sensitivities with respect to deformation of the optical fiber. Each Bragg grating is reacting different with respect to deformation of the optical fiber.

According to an embodiment, the first Bragg grating comprises a first sensitivity that is different to its second sensitivity, and wherein this second sensitivity is different to a third sensitivity of the first Bragg grating.

Furthermore, in an embodiment, the second Bragg grating comprises a second sensitivity that is different (e.g. larger) than its first sensitivity, and wherein the second sensitivity of the second Bragg grating is different (e.g. larger) than a third sensitivity of the second Bragg grating. In one embodiment, the first sensitivity of the second Bragg grating may be larger than the second sensitivity of the second Bragg grating that can be equal to the third sensitivity of the second Bragg grating.

Furthermore, particularly, the third Bragg grating comprises a third sensitivity that is different (e.g. larger) than its first sensitivity that can be equal to a second sensitivity of the third Bragg grating.

According to a further embodiment, the optical fiber comprises a fourth Bragg grating for measuring a temperature, wherein a portion of the optical fiber that comprises the fourth Bragg grating is surrounded by a protection tube arranged in the distal end portion of the shaft body. The optical fiber may be configured to move freely with respect to the protection tube.

Particularly, according to an embodiment, the four Bragg gratings are spaced apart from one another in the direction of the longitudinal axis of the shaft body. Particularly, the higher the number of the Bragg grating, the closer the respective Bragg grating is arranged to a distal end of the shaft body

Further, according to an embodiment, the optical fiber is fixed (e.g. glued) to an inner side of the first lumen in the region of the distal end portion of the shaft body. Particularly, the optical fiber comprises a cladding covering at least the Bragg gratings. The cladding can be formed from a heat-shrinkable tubing.

According to a further embodiment, the shaft body comprises a second lumen extending along the first lumen (or along the longitudinal axis of the shaft body), wherein a pulling wire for deflecting the shaft body is arranged in the second lumen.

Preferably, in an embodiment, the pulling wire is fixed (e.g. glued) to the distal end portion of the shaft body. Particularly, the pulling wire can be fixed (e.g. glued) to an inner sider of the second lumen in the region of the distal end portion of the shaft body to decouple a force measured with the optical fiber from a deflection of the shaft body of the catheter.

Furthermore, in an embodiment, for stiffening the pulling wire, a wire strand or a wire braid is arranged in the second lumen and extends in the distal end portion of the shaft body.

According to an embodiment, the catheter comprises a plurality of ring electrodes arranged on the distal end portion of the shaft body, wherein preferably each ring electrode is electrically connected to an electrical conductor extending in the shaft body towards a proximal end of the shaft body.

Furthermore, according to an embodiment, the catheter comprises a head electrode forming the catheter tip, wherein preferably the head electrode is electrically connected to an electrical conductor extending in the shaft body towards the proximal end of the shaft body. Preferably, according to an embodiment, the head electrode is fixed (e.g. glued) to a distal end of the shaft body (i.e. to a distal end of the distal end portion of the shaft body).

Furthermore, according to an embodiment, the catheter can also comprise an elongated temperature sensor (e.g. in form of a thermocouple) that is arranged in the shaft body.

According to a further embodiment, the catheter comprises a purging hose (e.g. irrigation hose) extending in the shaft body for purging the catheter. One or more openings may be formed in the distal portion of the catheter for letting the irrigation fluid out of the purging hose.

Particularly, in an embodiment, the catheter can comprise a rigid guiding tube arranged in the second lumen in the region of the distal end portion of the shaft body, wherein the guiding tube is inserted in the head electrode. Particularly, in a proximal direction, the guiding tube does not extend past the most proximal Bragg grating (first Bragg grating) or past the most proximal ring electrode of the catheter. Particularly, the purging hose passes through the guiding tube.

Particularly, in an embodiment, the catheter can comprise two lumens, i.e., the first and the second lumen, wherein the first lumen preferably comprises an inner diameter that is larger than an inner diameter of the second lumen.

In an embodiment, the electrical conductors that connect to the ring/head electrodes are arranged in the first lumen adjacent the optical fiber.

Furthermore, the elongated temperature sensor (e.g. thermocouple) can also be arranged in the first lumen adjacent said electrical conductors and optical fiber.

Furthermore, also the purging hose (and particularly a section of the guiding tube) can be arranged in the first lumen.

Preferably, at least a portion of the first lumen in the region of the distal end portion of the shaft body is filled with a glue to fix the electrical conductors, optical fiber, purging hose, and particularly also the temperature sensor to one another and to the distal end portion of the shaft body.

Furthermore, in an alternative embodiment, besides the first and the second lumen, the catheter can comprise a third and a fourth lumen, wherein the second lumen preferably comprises an inner diameter that is larger than the inner diameter of the first, third, and fourth lumen. According to an embodiment, the electrical conductors that are electrically connected to the ring electrodes are now preferably arranged in the third lumen, and the electrical conductor that is electrically connected to the head electrode is now preferably arranged in the fourth lumen.

Particularly, in case of the four-lumen catheter, the temperature sensor is preferably arranged in the third lumen. According to a further embodiment, when four lumens are present, the purging hose is preferably arranged in the second lumen (like the pulling wire, see above).

According to yet another embodiment, the optical fiber extends into the head electrode to allow light to exit from the optical fiber into an interior space of the head electrode or to allow light to exit from the head electrode. In the latter case, the optical fiber can extend through the head electrode. Here, the optical fiber can also be used to analyze tissue/blood of the patient or can be used for laser ablation (in case laser light is allowed to exit the head electrode via the optical fiber). Light reflected by blood and/or tissue may reenter the optical fiber. The reflected light may be processed (e.g. by a data processing unit connected to the catheter) in order to determine physiological parameters, e.g. oxygen saturation of the tissue. The catheter may be used as a fiber spectrometer.

In one embodiment, the catheter may comprise an elongated shaft body extending along a longitudinal axis and having a distal end portion connected to a catheter tip at a distal end of the catheter, wherein the shaft body comprises a first lumen extending along the longitudinal axis. The catheter further comprises an optical fiber, wherein the optical fiber extends in the first lumen, wherein particularly the first lumen extends along the longitudinal axis. In this embodiment, the optical analytics of blood or tissue is independent of the force measuring device. A catheter can be provided without Bragg gratings and consequently without force measuring function.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further features and embodiments of the present invention are described in the following with respect to the Figures, wherein:

FIG. 1A shows a distal end portion of a shaft body of a catheter comprising four lumens;

FIG. 1B shows a schematical cross-section along the longitudinal axis of the catheter shown in FIG. 1A;

FIG. 2 shows a schematical cross-section of the catheter shown in FIGS. 1A and 1B perpendicular to said longitudinal axis;

FIG. 3 shows a schematic illustration of the optical fiber of the catheter shown in FIGS. 1 to 2 that is used for measuring a force acting on the catheter tip;

FIG. 4 shows a schematical cross-section of a further embodiment of a catheter, wherein the shaft body of the catheter comprises two lumens;

FIG. 5 shows a schematical cross-section of the catheter shown in FIG. 4 along the longitudinal axis of the catheter;

FIG. 6 shows a further schematical cross-section of an embodiment of a catheter along the longitudinal axis of the catheter, wherein the optical fiber of the catheter extends through the head electrode of the catheter so that light can be radiated from an end of optical fiber out of the head electrode;

FIG. 7 shows an alternative detail of the cross-section shown in FIG. 4, wherein here the catheter comprises several optical fibers;

FIGS. 8A-8B show alternative configurations of the head electrode shown in FIG. 6, wherein according to FIG. 8A three optical fibers extend through the head electrode, and wherein according to FIG. 8B the optical fiber is glued into the head electrode, wherein the cured glue forms an optical element via which light passed through the optical fiber can exit the optical fiber at the end of the head electrode;

FIG. 9 shows a schematic illustration of the catheter shown in FIG. 6 and a measuring device connected to the optical fiber of the catheter;

FIG. 10 shows a further embodiment of the measuring device that is connected to a catheter of the kind shown in FIG. 8a comprising three optical fibers;

FIG. 11 shows a further embodiment of the measuring device that is connected to a catheter of the kind shown in FIG. 6, wherein here a force measuring unit, a spectrometer, and a light source (e.g. laser) are connected to the single optical fiber via a multiplexer;

FIG. 12 shows a further embodiment of a catheter, wherein here the optical fiber comprises an end portion that extends through the head electrode at an acute angle with respect to the longitudinal axis of the catheter; and

FIG. 13 shows a modification of the embodiment shown in FIG. 12, wherein here an end of the optical fiber is arranged in an interior space of the head electrode.

### DETAILED DESCRIPTION

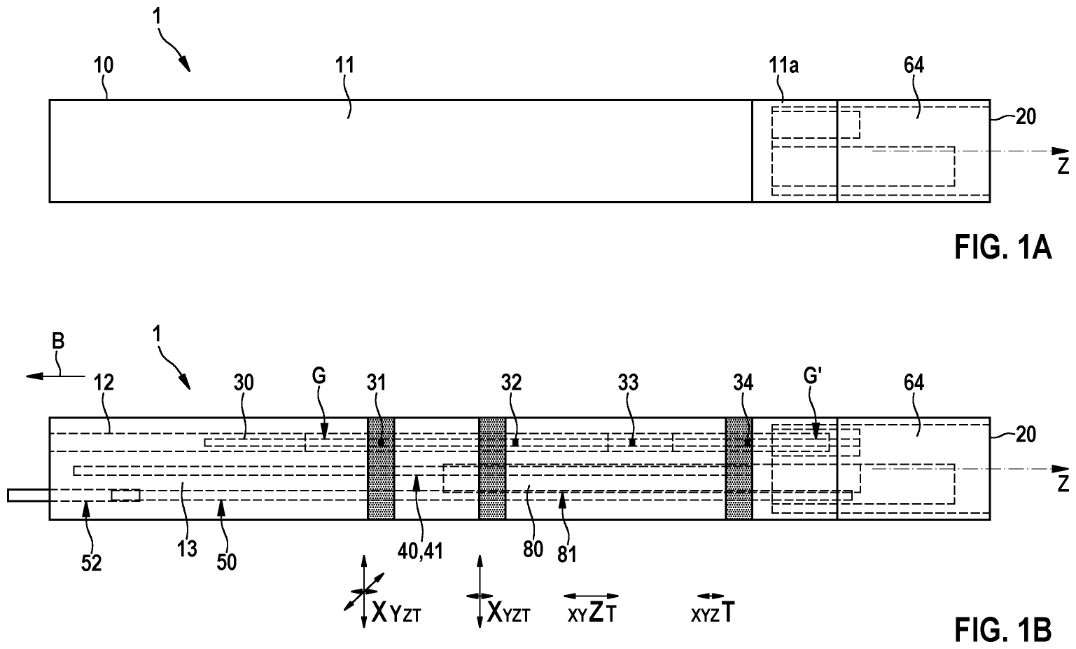

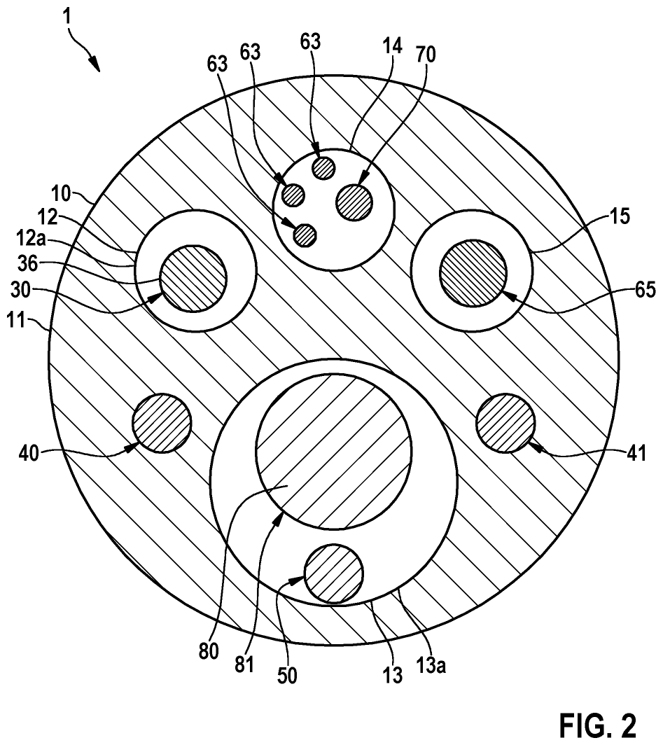

FIG. 1A shows, in conjunction with FIG. 1B and FIG. 2, an embodiment of a catheter 1. Such a catheter 1 can be used for ablation of tissue of a patient during a surgical procedure.

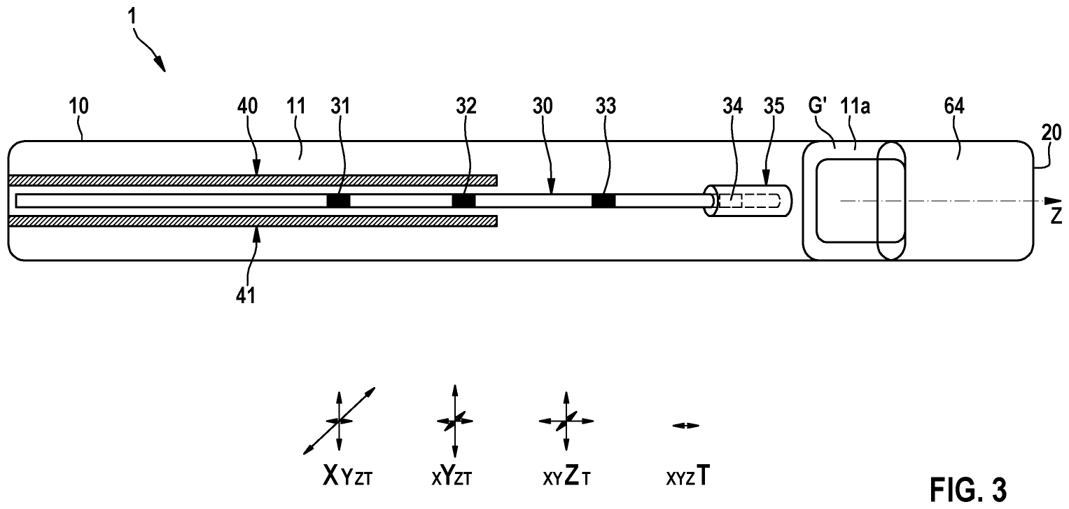

The catheter 1 comprises an elongated shaft body 10 extending along a longitudinal axis Z and having a distal end portion 11 connected to a catheter tip 20 at a distal end of the catheter 1, wherein the shaft body 10 comprises a first lumen 12, a second lumen 13, a third lumen 14, and a fourth lumen 15 (cf. FIG. 2) extending parallel to one another along the longitudinal axis Z. The catheter tip 20 is formed by a head electrode 64 that is preferably glued via a glue connection G′ to a distal end 11a of said portion 11 of the shaft body 10. The catheter further comprises e.g. three ring electrodes 60, 61, 62 arranged on the distal end portion 11 of the shaft body 10 of the catheter 1. Furthermore, the catheter 1 comprises an optical fiber 30 for measuring a force, wherein the optical fiber 30 extends in the first lumen 12 and preferably comprises a first, second, third, and a fourth Bragg grating 31, 32, 33, 34, wherein the first the second and the third Bragg grating 31, 32, 33 are configured for measuring a force acting on the catheter tip 20. Particularly, the fourth Bragg grating 34 serves for measuring a temperature in the vicinity of the head electrode 64 of the catheter 1. Preferably, the fourth Bragg grating 34 is arranged in a protection tube 35 that is embedded in the distal end portion 11 of the shaft body 10 as shown in FIG. 3 and is allowed to move freely with respect to the protection tube. This largely prevents a pressure load of the fourth Bragg grating so that a deformation of the latter is largely due to a varying temperature. In another embodiment, the fourth Bragg grating 34 may be completely covered with glue which is easier to manufacture.

Preferably, the catheter 1 does not comprise a metallic tubular force transducer for measuring a force acting on the catheter tip 20, but preferably comprises at least one less rigid component such as a first stiffening element 40 in the form of an elongated wire strand or an elongated wire braid to stiffen the distal end portion of the shaft body of the catheter 1. Preferably, the catheter also comprises a second stiffening element 41 in the form of a wire strand or wire braid as indicated in FIG. 2. Particularly, also the second stiffening element 41 is embedded in the distal end portion 11 of the shaft body for stiffening of the latter.

Particularly, the first stiffening elements 40, 41 extend along the longitudinal axis Z inside the distal end portion 11 parallel to the lumens 12, 13, 14, 15 of the shaft body for stiffening the distal end portion 11 of the shaft body 10.

Furthermore, as indicated in FIGS. 1B and 2 the catheter comprises a rigid guiding tube 81 of defined length that is fixed and inserted into the head electrode 62 and protrudes into the second lumen 13 in the region of the distal end portion 11 of the shaft body 10. Particularly the catheter may comprise a purging hose extending through the guiding tube 81, which purging hose 80 is configured for purging the catheter tip 20/head electrode 64 of the catheter. Furthermore, a pulling wire 50 for deflecting the shaft body 10 of the catheter 1 can be arranged in the second lumen 13. Particularly, as shown in FIG. 1B, the pulling wire 50 can be guided by a tubular pulling wire guide 52 that is arranged in the second lumen 13, too. Preferably, the second lumen 13 comprises a larger inner diameter than the other lumens 12, 14, 15. Particularly, the third lumen 14 can be utilized for accommodation of electrical conductors 63 that are used to electrically contact the ring electrodes 60, 61, 62. Furthermore, optionally, the third lumen 14 can accommodate an elongated temperature sensor 70 such as a thermocouple. Furthermore, particularly, the fourth lumen 15 can accommodate an electrical conductor 65 to electrically contact the head electrode 64.

In the area of the Bragg gratings 31 to 34, the optical fiber 30 is preferably arranged in the cladding 36, e.g. wrapped with shrinkable tube material so that a precise bonding is possible inside the first lumen 12. Preferably, apart from the region in which the third Bragg grating 33 for measuring a force component in the direction of the longitudinal axis Z is arranged (cf. FIG. 1B), the portions of the optical fiber 30 comprising the other Bragg gratings 31, 32, 34 are preferably glued to an inner side 12a of the first lumen by means of two glue connections G indicated in FIG. 1B. Furthermore, the head electrode 64 is glued to the distal end 11a of the distal end portion 11 of the shaft body 10 by means of a glue connection G′.

Particularly, the Bragg gratings 31, 32, 33, 34 are spaced apart from one another in the direction of the longitudinal axis Z of the shaft body 10 of the catheter 1, wherein particularly the Bragg gratings 31, 32, 33 comprise different sensitivities with regard to deformations of the optical fiber 30 in the direction of the longitudinal axis Z and the two orthogonal directions X and Y that extend perpendicular to the longitudinal axis Z of the shaft body 10 of the catheter 1 (see also above). This allows one to calculate the force components of a force acting on the catheter tip 20 by analyzing the wavelength shifts of the Bragg gratings 31, 32, 33 in a known manner.

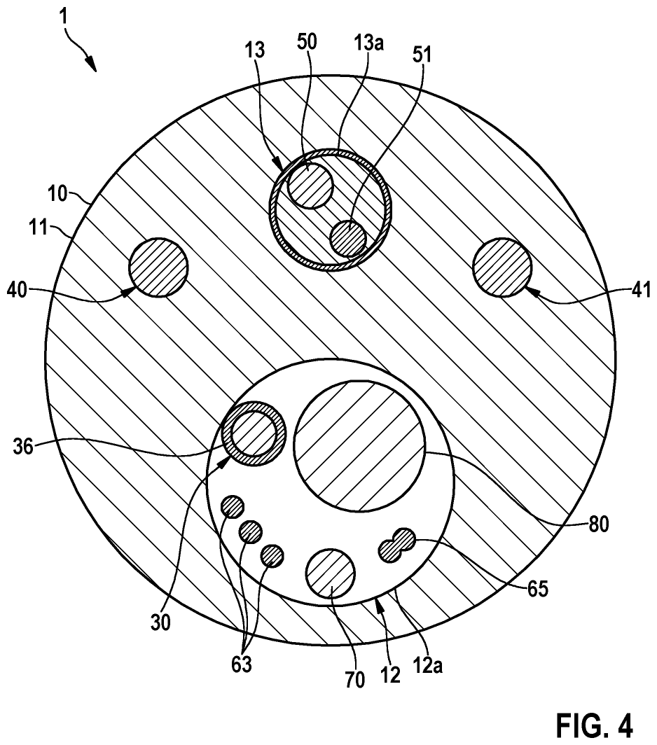

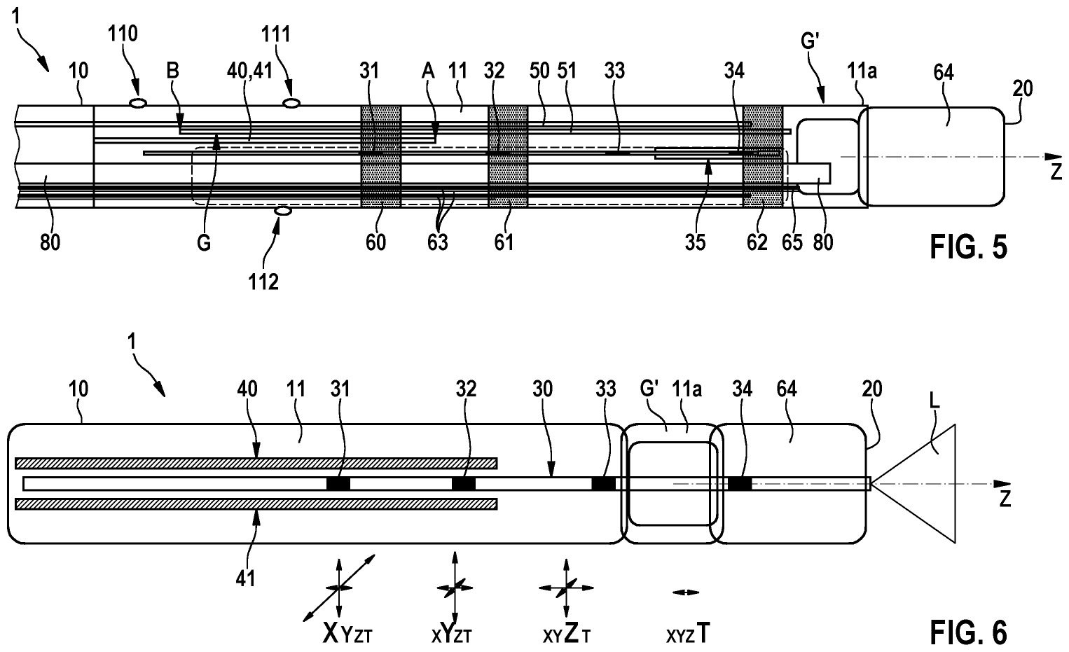

Furthermore, FIG. 4 shows, in conjunction with FIG. 5, a further embodiment of a catheter 1, wherein here the catheter 1 comprises merely two lumens, namely a first lumen 12 and a second lumen 13, wherein the first lumen 12 preferably comprises a larger inner diameter than the second lumen 12.

Also here, the optical fiber 30 is arranged in the first lumen 12. In contrast to the embodiment described above, the first lumen 12 also accommodates the electrical conductors 63 for electrically contacting the ring electrodes 60, 61, 62, the optional temperature sensor 70, and the electrical conductor 65 for making electrical contact to the head electrode 64. Furthermore, also the purging hose 80 can be accommodated in the first lumen 12 of the shaft body 10. The pulling wire 50 is separated from the other components and is arranged in a second lumen 13, preferably together with a stiffening element 51 in the form of the wire strand or a wire braid.

Preferably, the pulling wire 50 is glued to the distal end portion 11 of the shaft body 10, namely to an inner side 13a of the second lumen 13 in the region of the distal end portion 11 of the shaft body 10 to decouple a force measured with the optical fiber 30 from a deflection of the shaft body 10 of the catheter 1.

Also, in the embodiment shown in FIGS. 4 and 5, the catheter 1 comprises three ring electrodes 60, 61, 62 arranged on the distal end portion 11 of the shaft body 10 and connected to the respective electrical conductor 63 (cf. FIG. 5), as well as a head electrode 64 forming the catheter tip 20, wherein the head electrode 64 is electrically connected to said electrical conductor 65. Also here, the head electrode 64 is preferably glued via a glue connection G′ to the distal end 11a of the shaft body 10/distal end portion 11.

Further, for stiffening the distal end portion 11 of the shaft body 10, the catheter 1 preferably comprises a first and a second stiffening element 40, 41 in the form of a wire strand or a wire braid which extend parallel with respect to one another and are embedded in the distal end portion 11 of the shaft body 10 of the catheter 1 (cf. FIG. 4).

Furthermore, as shown in FIG. 5, the optical fiber 30 can be configured as described above and may comprise a first, second, third, and a fourth Bragg grating 31, 32, 33, 34, wherein the fourth Bragg grating 34 is preferably arranged in a protection tube 35 as described above.

According to an example shown in FIG. 5, particularly in case the head electrode 64 of the catheter 1 comprises an outer diameter of 7F (i.e. 2.67 mm), the first Bragg grating 31 can be positioned at a distance of 10 mm to the distal end 11a of the shaft body 10/distal end portion 11 of the catheter 1. Furthermore, this distance can amount to 7 mm for the second Bragg 32 grating, 4 mm for the third Bragg grating 33 and 1 mm for the fourth Bragg grating 34. Furthermore, according to the specific example shown in FIG. 5, the stiffening elements 40, 41 can extend from point B to point A along the longitudinal axis Z of the shaft body 10 of the catheter 1, wherein point B can be spaced apart 15 mm from said distal end 11a, and wherein point A can be spaced apart 8 mm from said distal end 11a.

Furthermore, the distal end portion 11 of the shaft body 10 may comprise lateral openings 110, 111 for inserting the stiffening elements 40, 41, 51 (e.g. wire strand or wire braid) into the distal end portion 11 of the shaft body 10 and for applying glue to the pulling wire 50 in the second lumen 13 to achieve a glue connection G for fixing the pulling wire 50 in the second lumen 13 (see also above). According to a specific example, the stiffening elements 40, 41 may extend from a starting point being positioned 11 mm from the distal end 11a apart towards the distal end 11a of the shaft body 10.

Furthermore, a glue can be applied through a lateral opening 112 of the distal end portion 11 of the shaft body 10 so as to fill the first lumen 12 starting from the position of the lateral opening 112 up to the distal end 11a of the shaft body 10 with said glue to establish a glue connection G″ for fixing the components 30, 63, 65, 70 arranged in the first lumen 12 with respect to the distal end portion 11 of the shaft body 10. According to a specific example, the glue connection G″ can have an extension of 12 mm long the longitudinal axis Z.

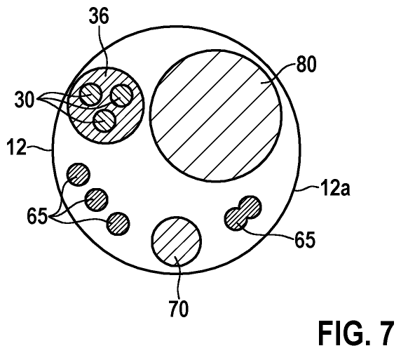

Furthermore, FIGS. 6 to 13 show embodiments where the catheter 1 comprises at least one optical fiber 30 that extends into the head electrode 64 to allow light L to exit from the optical fiber 30 into an interior space 64a (cf. FIG. 13) of the head electrode 64 or to allow light L to exit from the head electrode 64. In the latter case, the optical fiber 30 can extend through the head electrode 64.

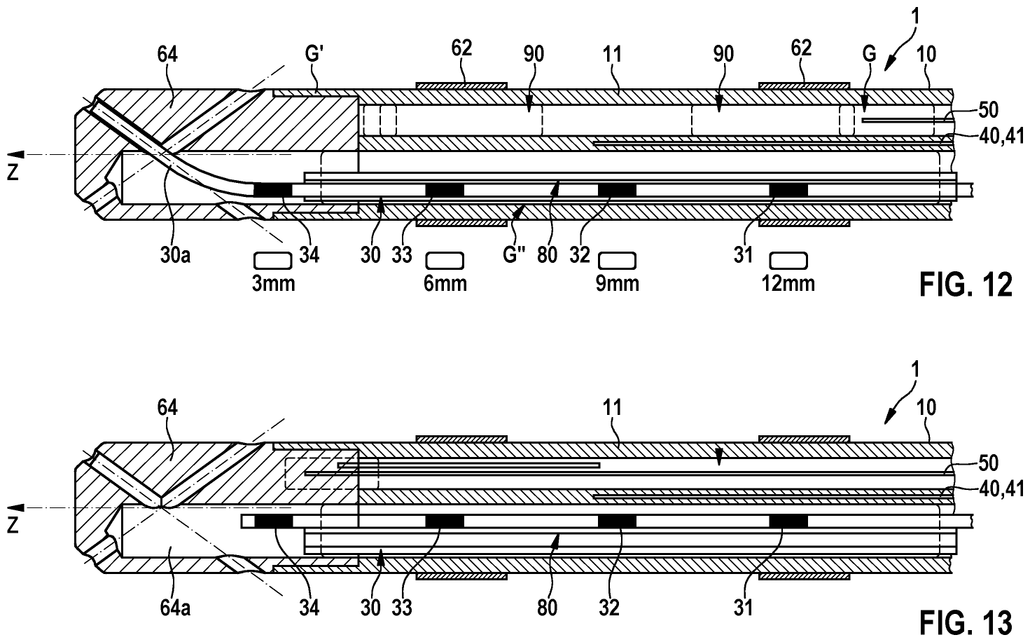

If the optical (e.g. glass) fiber 30 is optically guided up to the catheter tip 20, particularly through the head electrode 64, light L can exit distally (e.g. diffuse) and the tissue can be analyzed using reflected light. Particularly, a real-time measurement of oxygen saturation of blood of the patient (e.g. in the heart chamber), spectroscopy of blood or tissue of the patient in vivo, sclerotherapy of tissue by laser ablation as well as stimulation of the tissue by light (e.g. pulse) can be carried out using a configuration of the optical fiber 30 as shown in FIG. 6. Furthermore, as indicated in FIG. 12, the optical fiber 30 can comprise an end section 30a extending in the head electrode 64 that is arranged at an angle with respect to the longitudinal axis Z of the catheter 1/shaft body.

Particularly, the oxygen content can be determined by relative measurement at different wavelengths, wherein relative measurements over a wavelength spectrum are independent of dilution by catheter flushing. Furthermore, the IR spectrum used can be adapted to the area to be analyzed.

Alternatively, as shown in FIG. 13, the optical fiber 30 may also end in the flushed area, i.e. interior space 64a, inside the head electrode 64. This would allow measurement of the integral reflected light. Particularly, also the water column during rinsing could be used as a light guide (i.e. as a supplement or replacement for an optical fiber 30, e.g. for lighting).

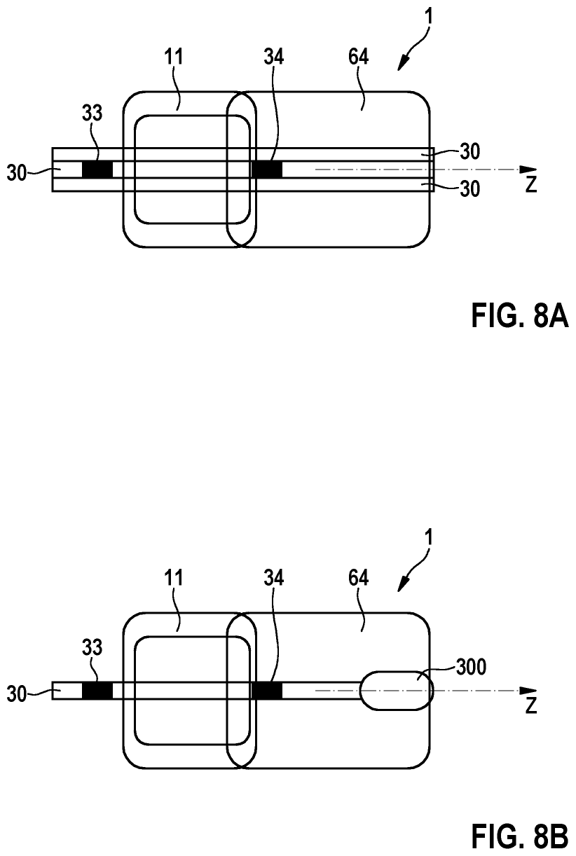

According to the embodiment shown in FIG. 8A more than one optical fiber 30, particularly three optical fibers 30, extend to the distal end/catheter tip 20 of the catheter 1 and are bonded to the head electrode 64. Here one optical fiber 30 can comprise Bragg gratings 33, 34, . . . and is used for measuring a force acting on the catheter tip 20. The other two adjacent optical fibers 30 can be used for optical spectroscopy and light transmission. Using e.g. three optical fibers 30 allows to physically separate the force measuring function, optical spectroscopy and light transmission from one another.

According to the embodiment shown in FIG. 8B, a single optical (e.g. glass) fiber can be passed into the head electrode 64 and can be fixed there using an adhesive. Particularly, the adhesive may fill a front cavity of the head electrode 64 and form an optical element 300 (e.g. in form of a lens or a diffusor). Furthermore, the adhesive can also act as a mechanical damper.

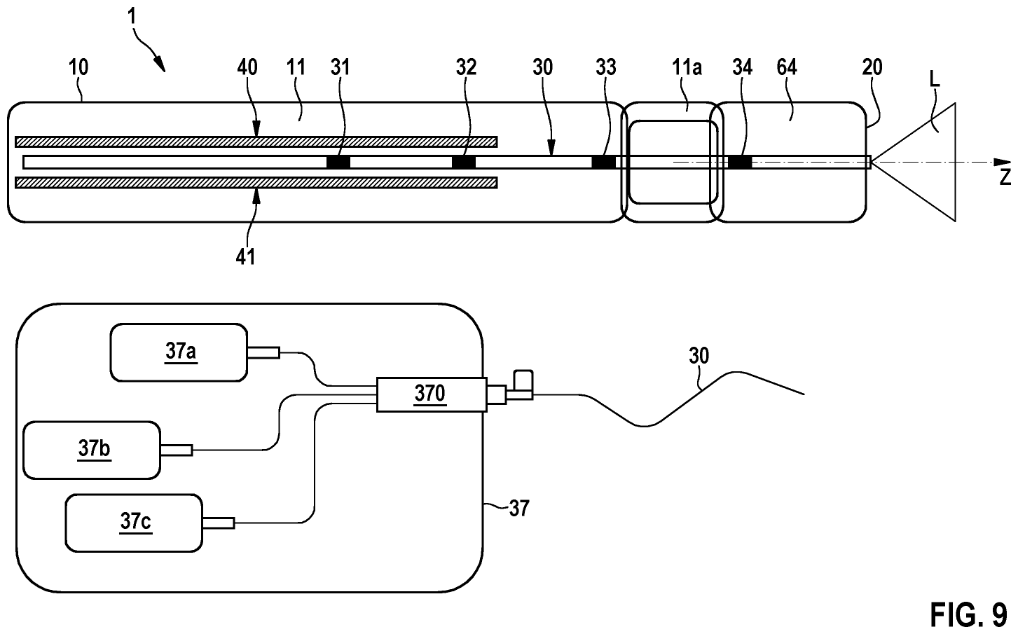

Particularly, FIG. 9 shows an embodiment of a catheter 1 having an optical fiber 30 that extends through the head electrode 64 so that light L can exit the head electrode 64, wherein the catheter 1 comprises a measuring device 37 that comprises a beam splitter 370 to connect the single optical fiber 30 to a force measuring unit 37a, to a spectrometer 37b, and to a light source (e.g. laser) 37c for emitting light into the optical fiber 30. Thus, according to the embodiment shown in FIG. 9, all signals are routed through the same optical fiber 30.

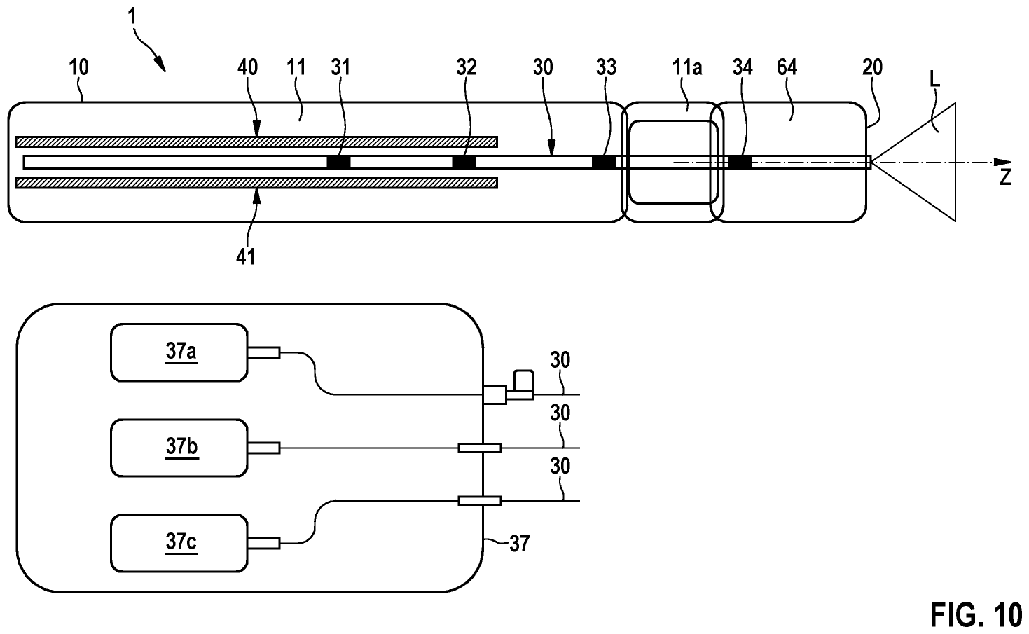

Alternatively, as shown in FIG. 10, the force measuring unit 37a, the spectrometer 37b, and the light source (e.g. laser) 37c are each connected an associated optical fiber of said three optical fibers 30 (cf. also FIG. 8A).

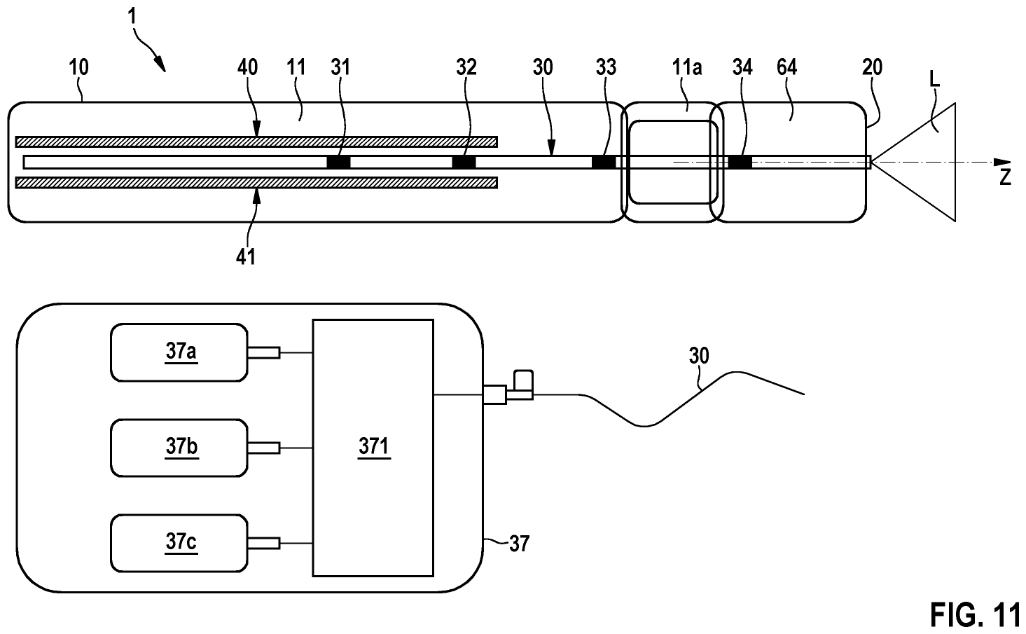

Furthermore, according to the embodiment shown in FIG. 11, a single optical fiber 30 may also be used that is connected via a multiplexer/chopper/frequency modulating device 371 of the measuring device 37 to the force measuring unit 37a, to the spectrometer 37b, and to the light source (e.g. laser 37c). Thus, also here, the same optical fiber 30 can be used for different applications.

The catheter design according to the present disclosure enables a number of different advantages. Particularly, the applicability is improved since the catheter 1 according to the various embodiments allows reversible deformations in the tip area (e.g. caused by an inward sluice).

Furthermore, the elimination of the rigid force transducer greatly reduces manufacturing costs and enables the construction of thinner catheters.

By using the optical fiber with Bragg gratings, force measurements in all spatial directions can be performed. Furthermore, the optical fiber can also be used for oxygen measurements, spectral evaluations, chemical analysis, light application for stimulation (e.g. with low energy supply for the stimulation of chemical or physical processes), and laser ablation.

Particularly, evaluation of the relative spectral changes e.g.: around 660 nm and 900 nm result in information about oxygen saturation (haemoglobin complex). Advantageously, evaluation of oxygen saturation can be correlated with tissue properties.

Particularly, in case of using choppers/login amplifiers or other frequency modulation, assessing further effects in different tissue depths (e.g. phosphorescence etc.) is possible.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A catheter, comprising:
an elongated shaft body extending along a longitudinal axis and having a distal end portion connected to a catheter tip at a distal end of the catheter, wherein the shaft body comprises a first lumen extending along the longitudinal axis, and
an optical fiber for measuring a force, wherein the optical fiber extends in the first lumen and comprises at least a first Bragg grating arranged in the distal end portion of the shaft body,
wherein the distal end portion of the shaft body encloses at least a first stiffening element, wherein the first stiffening element extends along the longitudinal axis for stiffening the distal end portion of the shaft body.

2. The catheter according to claim 1, wherein the first stiffening element is in the form of an elongated wire strand, an elongated wire braid, an elongated tubing or a flat spring.

3. The catheter according to claim 1, wherein the distal end portion of the shaft body encloses a second stiffening element, wherein the second stiffening element extends along the longitudinal axis for stiffening the distal end portion of the shaft body.

4. The catheter according to claim 1, wherein for measuring said force acting on the catheter tip, the optical fiber comprises a second Bragg grating formed in a portion of the optical fiber, wherein the second Bragg grating is arranged in the distal end portion of the shaft body, and wherein the optical fiber comprises a third Bragg grating formed in a portion of the optical fiber, wherein the third Bragg grating is arranged in the distal end portion of the shaft body.

5. The catheter according to claim 1, wherein the optical fiber comprises a fourth Bragg grating for measuring a temperature, wherein a portion of the optical fiber that comprises the fourth Bragg grating is surrounded by a protection tube arranged in the distal end portion of the shaft body and configured to move freely with respect to the protection tube.

6. The catheter according to claim 1, wherein the shaft body comprises a second lumen, wherein a pulling wire for deflecting the shaft body is arranged in the second lumen.

7. The catheter according to claim 6, wherein the pulling wire is fixed to the distal end portion of the shaft body.

8. The catheter according to claim 6, wherein for stiffening the pulling wire a further stiffening element in the form of a wire strand or a wire braid is arranged in the second lumen and extends in the distal end portion of the shaft body.

9. The catheter according to claim 1, wherein the catheter comprises a plurality of ring electrodes arranged on the distal end portion of the shaft body, wherein particularly each ring electrode is electrically connected to an electrical conductor extending in the shaft body.

10. The catheter according to claim 1, wherein the catheter comprises a head electrode forming the catheter tip, wherein particularly the head electrode electrically connected to an electrical conductor extending in the shaft body.

11. The catheter according to claim 1, wherein the catheter comprises a purging hose extending in the shaft body for purging the catheter.

12. The catheter according to claim 9, wherein the electrical conductors are arranged in the first lumen.

13. The catheter according to claim 11, wherein the purging hose is arranged in the first lumen.

14. The catheter according to claim 9, wherein the catheter comprises a third and a fourth lumen, wherein the electrical conductors that are electrically connected to the ring electrodes are arranged in the third lumen, and wherein the electrical conductor that is electrically connected to the head electrode is arranged in the fourth lumen.

15. The catheter according to claim 1, wherein the optical fiber extends into the head electrode to allow light to exit from the optical fiber into an interior space of the head electrode or to allow light to exit from the head electrode.
